(19) United States
(12) Patent Application Publication
(10) Pub. No.: US 2024/0188801 A1
(43) Pub. Date: Jun. 13, 2024

(54) DIAGNOSTIC INSTRUMENT AND A MEDICAL DEVICE ASSEMBLY INCLUDING THE SAME

(71) Applicant: Prisma Health, Greenville, SC (US)

(72) Inventors: Daniel LoVerde, Greenville, SC (US); Essam Z. Namouz, Greenville, SC (US)

(21) Appl. No.: 18/458,371

(22) Filed: Aug. 30, 2023

Related U.S. Application Data
(60) Provisional application No. 63/402,218, filed on Aug. 30, 2022.

(51) Int. Cl. A61B 1/00; A61B 1/018

(52) U.S. Cl. CPC A61B 1/00071; A61B 1/00121; A61B 1/018

(57) ABSTRACT

A diagnostic instrument is provided, including a proximal end; a penetrating component; and an attachment assembly positioned between the proximal end and the penetrating component to allow for separation of the proximal end from the penetrating component without fully retracting the penetrating component from a body of a patient during use of the diagnostic instrument.

### CROSS-REFERENCE TO RELATED APPLICATIONS

This application is a non-provisional application claiming the benefit of priority under 35 U.S.C. § 119(e) to U.S. Provisional Application No. 63/402,218, filed Aug. 30, 2022, which is hereby incorporated by reference in its entirety.

### FIELD

The present disclosure relates to a diagnostic instrument such as an endoscope or bronchoscope and a medical device assembly including the same.

### BACKGROUND

Diagnostic procedures involving body-insertable instruments are commonly used in the medical field. One such instrument is a bronchoscope, employed to visualize and access the airways. Often during a procedure utilizing a diagnostic instrument, it becomes essential to deploy a secondary device over a primary penetrating component of the diagnostic instrument. One of the challenges faced during such operations is the need to change a size or even a type of the secondary deployable device after initial insertion. The inventors of the present disclosure have found that conventional diagnostic instruments do not allow for safe detachment of a proximal end in a manner that facilitates a consistent exchange of secondary devices without completely withdrawing the penetrating component from the patient's body. The inventors have found that this limitation can lead to prolonged procedure durations, potential health risks to patients, and may compromise the success of certain difficult procedures.

Accordingly, the inventors of the present disclosure have found that a need exists for an improved approach to diagnostic procedures that allows for quick and safe exchange of deployable devices without a need for full instrument removal and reinsertion.

### BRIEF DESCRIPTION OF THE DRAWINGS

A full and enabling disclosure of the present disclosure, including the best mode thereof, directed to one of ordinary skill in the art, is set forth in the specification, which makes reference to the appended figures, in which:

FIG. 1 provides a schematic view of a medical device assembly in accordance with an exemplary aspect of the present disclosure.

FIG. 2 provides a schematic view of the exemplary medical device assembly of FIG. 1, with an attachment assembly in a detached configuration.

FIG. 3 provides a perspective view of a diagnostic instrument in accordance with an exemplary aspect of the present disclosure.

FIG. 4 provides a schematic view of an attachment assembly of the diagnostic instrument of FIG. 3 in accordance with an exemplary aspect of the present disclosure.

FIG. 5 provides a perspective view of the attachment assembly of FIG. 4.

FIG. 6 provides a plan view of a second member of the attachment assembly of FIG. 4.

FIG. 7 provides a first cross-sectional view of the exemplary attachment assembly of FIG. 4, through line 7-7 in FIG. 4.

FIG. 8 provides a second cross-sectional view of the exemplary attachment assembly of FIG. 4, through line 7-7 in FIG. 4.

FIG. 9 provides a cross-sectional view of the exemplary attachment assembly of FIG. 4, through line 9-9 in FIG. 4.

FIG. 10 provides another perspective view of the attachment assembly of FIG. 4.

FIG. 11 provides a flow diagram of a method of operating a medical device assembly in accordance with an exemplary aspect of the present disclosure.

### DETAILED DESCRIPTION

The word “exemplary” is used herein to mean “serving as an example, instance, or illustration.” Any implementation described herein as “exemplary” is not necessarily to be construed as preferred or advantageous over other implementations. Additionally, unless specifically identified otherwise, all embodiments described herein should be considered exemplary.

The singular forms “a”, “an”, and “the” include plural references unless the context clearly dictates otherwise.

Often times body cavities, orifices or tubular structures of the body (e.g., the oropharynx, nasopharynx, trachea, bronchi, esophagus, small or large intestine, vascular structures, biliary tree or urethra) are accessed with a diagnostic instrument such as an endoscope or other similar body insertable diagnostic instrument. The diagnostic instrument is comprised of a proximal end and a penetrating end or penetrating component. Frequently these diagnostic instruments are used to facilitate the placement of a secondary deployable device that could include, but is not limited to: a tube, drain, wire or port. The technique for this is typically performed by loading the secondary device over the distal end of a penetrating component of the diagnostic instrument, then advancing the penetrating component of the diagnostic instrument into the desired location. This allows the secondary device to be properly positioned. An example of this is the use of a bronchoscope to deploy an endotracheal tube after the distal tip of the penetrating component of the bronchoscope is driven down into the trachea. However, other devices may use a similar technique.

Occasionally while attempting to deploy a secondary device over the penetrating component of a diagnostic instrument, it is determined that a different size deployable device is desired. When this occurs, the diagnostic instrument must be removed from the body so that the deployable device can be removed, and a new deployable device can be re-loaded. The diagnostic instrument must then be re-inserted into the body and repositioned before the deployable device can be advanced. This wastes time and can present risk to the patient. This is due to the fact that the proximal end of the diagnostic instrument cannot be safely detached from the tubing unit/penetrating component in a location that would allow the exchange of the secondary device without withdrawing the penetrating component from the patient's body.

Further to the example above, during difficult intubations, a bronchoscope that is pre-loaded with an endotracheal tube may be used to facilitate passage through a patient's vocal folds. If the endotracheal tube that was loaded onto the bronchoscope is the wrong size, the entire bronchoscope apparatus must be withdrawn from the patient so a different size endotracheal tube can be loaded. The bronchoscope (with new endotracheal tube) must then be reinserted, passed again through the vocal folds to deploy the correct size endotracheal tube. This presents several possible issues including: 1) in a critically narrowed airway it may not be possible to pass through the vocal folds again, 2) if access to the trachea is obtained again, there would have been a time delay in providing critical ventilatory support.

The present disclosure provides for a device that allows for separation of the diagnostic instrument between the proximal end and the penetrating component. The separation of the proximal end from the penetrating component allows a deployable component (also referred to herein as a secondary device) to be removed without having to withdraw the penetrating component of the diagnostic instrument from the body. This allows for an exchange of another secondary deployable device over the penetrating component such as, but not limited to a tube or other structure without removing the penetrating component of the diagnostic instrument from the patient's body.

An exemplary embodiment of the diagnostic instrument that would utilize the disclosure device is an endoscope, more specifically a bronchoscope. In a broad sense, a bronchoscope is a diagnostic instrument used to gain access to and visualize the inside of airways. The penetrating component of the bronchoscope is generally inserted into the patient's airway. A typical bronchoscope includes of a handle at the proximal end with a channel/tube/shaft that extends to the distal penetrating component. The channel may contain a light, a camera, a suction tube, a working channel, and a mechanism with which to articulate the tip of the penetrating component. The power and signal wires needed to operate various functions are also carried within the channel. In many bronchoscopes, a controllable lever located on the proximal end that when actuated by the operator, articulates the penetrating tip of the bronchoscope.

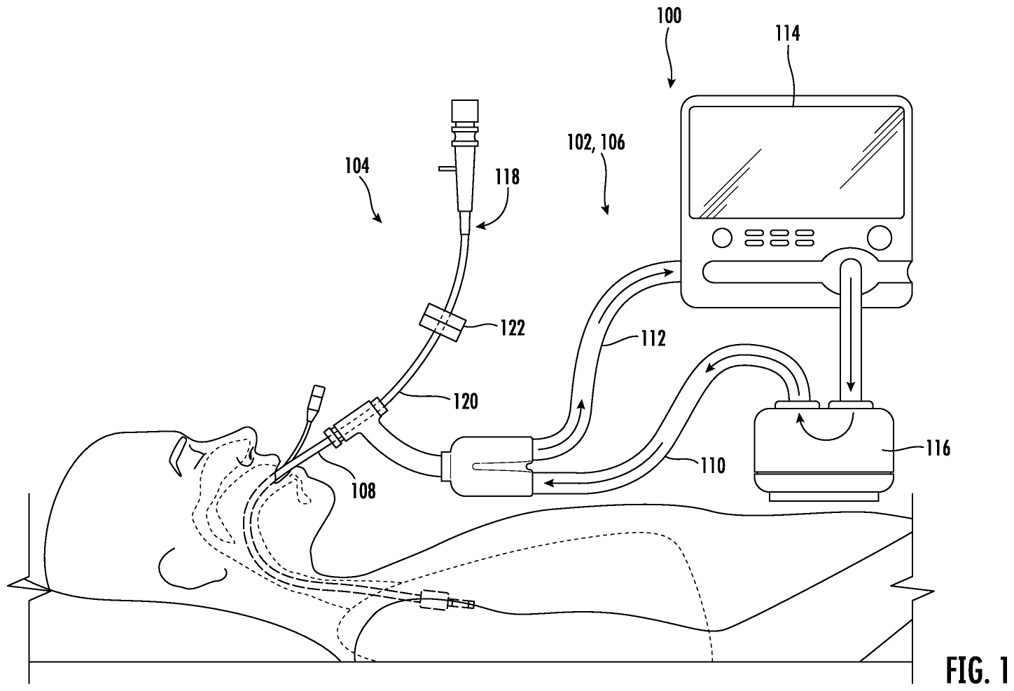

Referring now to the figures, FIG. 1 provides a schematic view of a medical device assembly 100 in accordance with an exemplary aspect of the present disclosure. The medical device assembly 100 generally includes a secondary device 102, also referred to herein as a deployable device, and a diagnostic instrument 104.

In the embodiment shown, the secondary device 102 is configured as a ventilator assembly 106 configured to provide airflow to, and receive airflow from, a patient. The ventilator assembly 106 includes an insertable tube 108, also known as an endotracheal tube, an airflow delivery tube 110, and an airflow exhaust tube 112. The ventilator assembly 106 further includes a ventilator 114 configured to extract air from the patient through the airflow exhaust tube 112 and provide airflow to the patient through the airflow delivery tube 110. In addition, the exemplary ventilator assembly 106 depicted includes a humidifier 116 to maintain a desired humidity in the airflow provided to the patient via the airflow delivery tube 110. The airflow delivery tube 110 is in connection with the endotracheal tube 108 to provide the airflow to lungs of the patient. The airflow exhaust tube 112 may be in airflow communication with a working gas path of the diagnostic instrument 104 (as will be discussed in more detail, below) to extract air from the lungs of the patient, or may be connected to a separate suction device (no shown; separate from the ventilator 114).

The diagnostic instrument 104 is, for the embodiment depicted, an endoscope, and includes a proximal end 118, a penetrating component 120, and an attachment assembly 122 positioned between the proximal end 118 and the penetrating component 120. As will be appreciated, for the embodiment shown, the penetrating component 120 extends at least partially through a tube of the secondary device 102 inserted into the patient, and more specifically, extends at least partially through the endotracheal tube 108 of the ventilator assembly 106.

The attachment assembly 122, as noted above, is positioned between the proximal end 118 and the penetrating component 120 to allow for separation of the proximal end 118 from the penetrating component 120 without fully retracting the penetrating component 120 from the body of the patient during use of the diagnostic instrument 104.

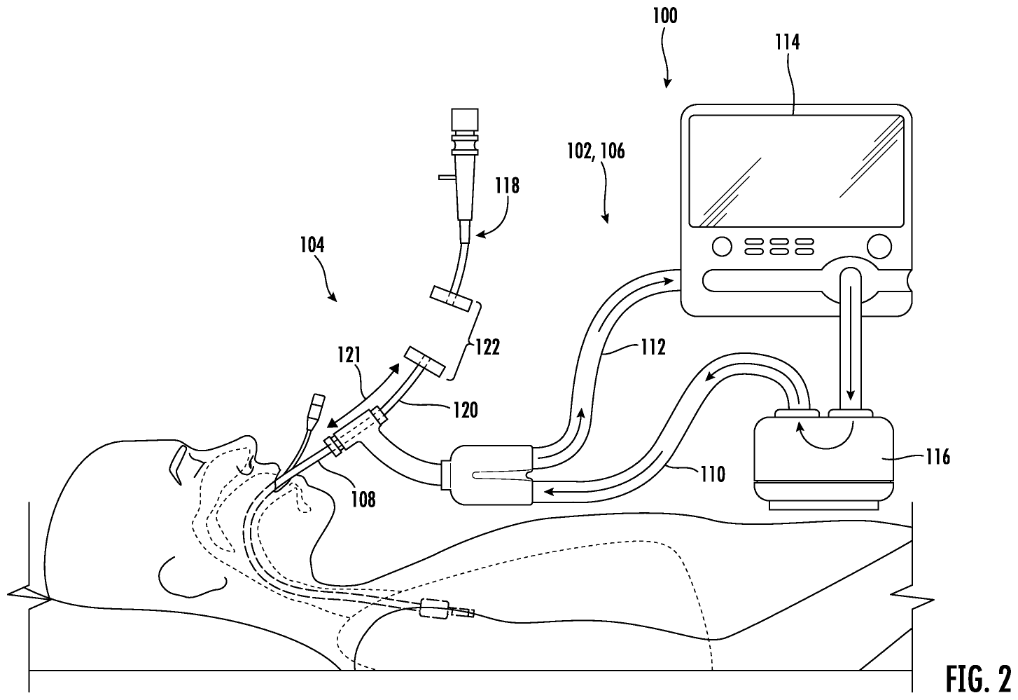

More specifically, referring now also to FIG. 2, providing another schematic view of the medical device assembly 100 of FIG. 1, it will be appreciated that the attachment assembly 122 is movable between an attached configuration (shown in FIG. 1) and a detached configuration (shown in FIG. 2). When the attachment assembly 122 is moved to the detached configuration, the tube 108 of the secondary device 102 may be removed from the patient without retracting the penetrating component 120 of the diagnostic instrument 104 from the body of the patient. A second tube of a different secondary device (not shown) may then be provided over the penetrating component 120 and inserted into the body of the patient, again without retracting the penetrating component 120 from the body of the patient. Such is indicated by the dual-sided arrow 121 in FIG. 2. Once the second tube of the different secondary device is provided over the penetrating component 120 and inserted into the body of the patient, the attachment assembly 122 may be moved back to the attached configuration (see FIG. 1), and all functionality of the diagnostic instrument 104 may be resumed, or at least a portion of the functionality of the diagnostic instrument 104 may be resumed. For example, in one example embodiment, one or all of a light functionality, a camera functionality, and an articulation functionality of the diagnostic instrument 104 may be resumed.

It will be appreciated that although the tube 108 of the secondary device 102 is an endotracheal tube and the diagnostic instrument 104 is an endoscope in the exemplary embodiment of FIGS. 1 and 2, as discussed above, in other embodiments, other suitable secondary devices and diagnostic instruments are contemplated by the present disclosure. For example, in other exemplary embodiments, the secondary device may be a laryngeal mask airway.

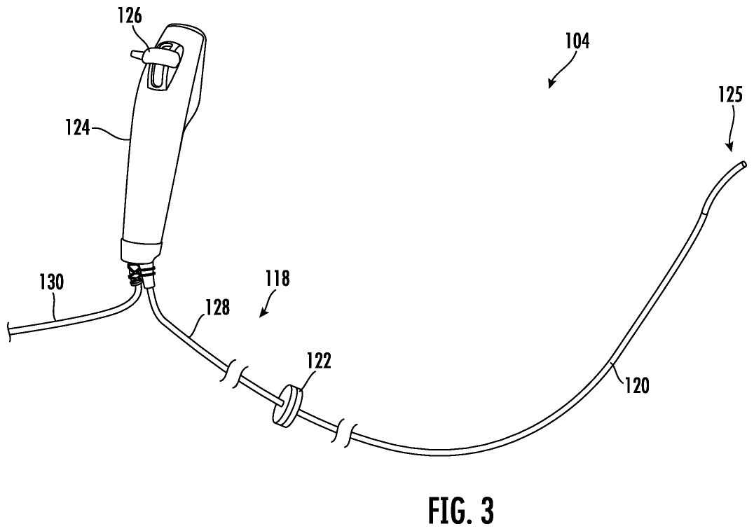

Referring now to FIG. 3, a diagnostic instrument 104 in accordance with an exemplary aspect of the present disclosure is provided. The diagnostic instrument 104 includes a proximal end 118, a penetrating component 120 configured to be received within a body of a patient during use, and an attachment assembly 122 positioned between the proximal end 118 and the penetrating component 120 to allow for separation of the proximal end 118 from the penetrating component 120 without fully retracting the penetrating component 120 from the body of the patient during use of the diagnostic instrument 104.

The proximal end 118 generally includes a handle 124 having a control lever 126. The control lever 126 may allow for actuation of a distal end 125 of the penetrating component 120. Although not depicted, the handle 124 may further include other switches, levers, ports, etc. for controlling various functionalities of the diagnostic instrument 104 (e.g., lights, cameras, suction airflow, etc.).

The proximal end 118 further includes a main line 128 and a supporting line 130. The supporting line 130 may be used to convey data to one or more additional components, may be configured to receive electric power from an electric power source, etc. For example, the supporting line 130 may be configured to provide a video feed of a camera located at the distal end 125 of the penetrating component 120, may be configured to receive electrical power (e.g., to provide power to a camera located at the distal end 125 of the penetrating component 120, to provide power to a light located at the distal end 125 of the penetrating component 120), or both. The main line 128 may have the same function as the supporting line 130 with the addition of one or more control wires or the like to physically actuate the distal end 125 of the penetrating component 120.

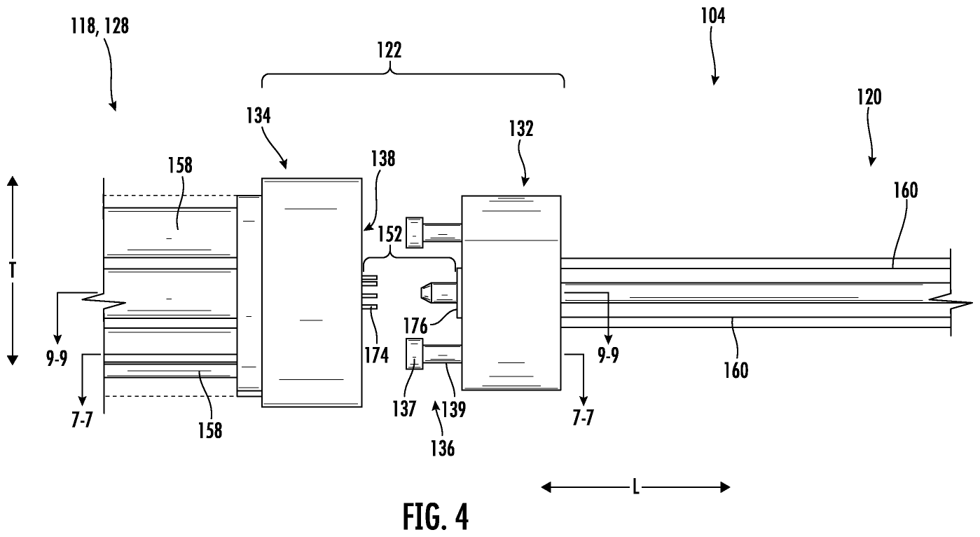

Referring now also to FIG. 4, a close-up view of the attachment assembly 122 of FIG. 3 is provided. The attachment assembly 122, as noted, is movable between an attached configuration (see FIG. 3) and a detached configuration (see, e.g., FIG. 4).

As will be appreciated from the view of FIG. 4, the attachment assembly 122 includes a first member 132 connected to the penetrating component 120 and a second member 134 connected to the proximal end 118. The first member 132 and the second member 134 are releasably attachable to one another. In particular, the first member 132 includes first attachment features 136 and the second member 134 includes second attachment features 138. The first attachment features 136 and the second attachment features 138 interlock when rotated relative to one another.

As will be appreciated from the description herein, a tube from a secondary device (e.g., an insertable tube 108 of a ventilator assembly 106; see FIGS. 1 and 2) may be removed over the first member 132 of the attachment assembly 122 and/or provided over the first member 132 of the attachment assembly 122 the when the attachment assembly 122 is in the detached configuration. In such a manner, it will be appreciated that the first member 132 may define a relatively small maximum width in a transverse direction T.

For example, in the embodiment depicted, the first attachment features 136 include pins with an enlarged distal end 137. For example, the exemplary attachment assembly 122 depicted defines a longitudinal direction L and a transverse direction T perpendicular to the longitudinal direction L. The exemplary pins extend generally along the longitudinal direction L, and the distal end 137 of each pin defines a thickness along the transverse direction T greater than a thickness of a body 139 of the pins.

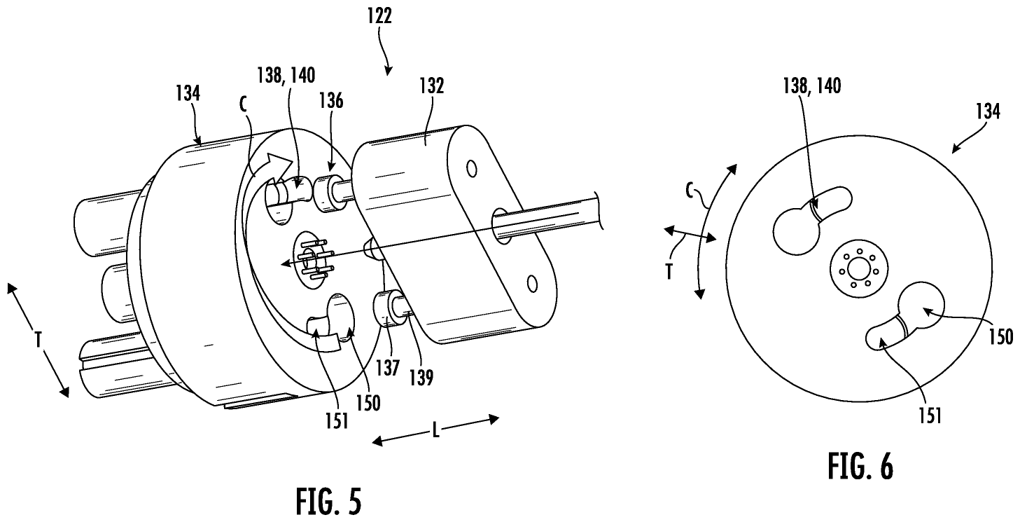

Referring now also to FIG. 5, which provides a perspective view of the exemplary attachment assembly 122 of FIG. 4, it will be appreciated that the second attachment features 138 include slots 140 defined by the second member 134 configured to receive the pins.

Referring now also to FIG. 6, which provides a plan view of an attachment surface of the second member 134, it will be appreciated that each slot 140 defines a main opening 150 and a narrow section 151. The main opening 150 defines a width equal to or greater than the thickness of the distal end 137 of a respective pin, and the narrow section 151 of each slot 140 defines a width less than the thickness of the distal end 137 of the respective pin and greater than or equal to the thickness of the body 139 of the respective pin. The widths and thicknesses discussed herein refer to measurements in the transverse direction T.

In such a manner, the first member 132 may be interlocked with the second member 134 by inserting the distal ends 137 of the pins into the main openings 150 of the respective slots 140 and rotating the first member 132 relative to the second member 134 in a circumferential direction C (i.e., a direction extending about the longitudinal direction L), such that the bodies of the respective pins are moved into the narrow sections 151 of the respective slots 140, preventing removal of the pins from the slots 140 and preventing removal of the first member 132 from the second member 134.

In the embodiment depicted, the first attachment features 136 include two pins, and the second attachment features 138 include two slots 140. It will be appreciated, however, that in other exemplary embodiments, the first attachment features 136 may have any other suitable number of pins (e.g., at least three and up to 10) and the second attachment features 138 may include any other suitable number of slots 140 defined by the second member 134 corresponding to the number of pins.

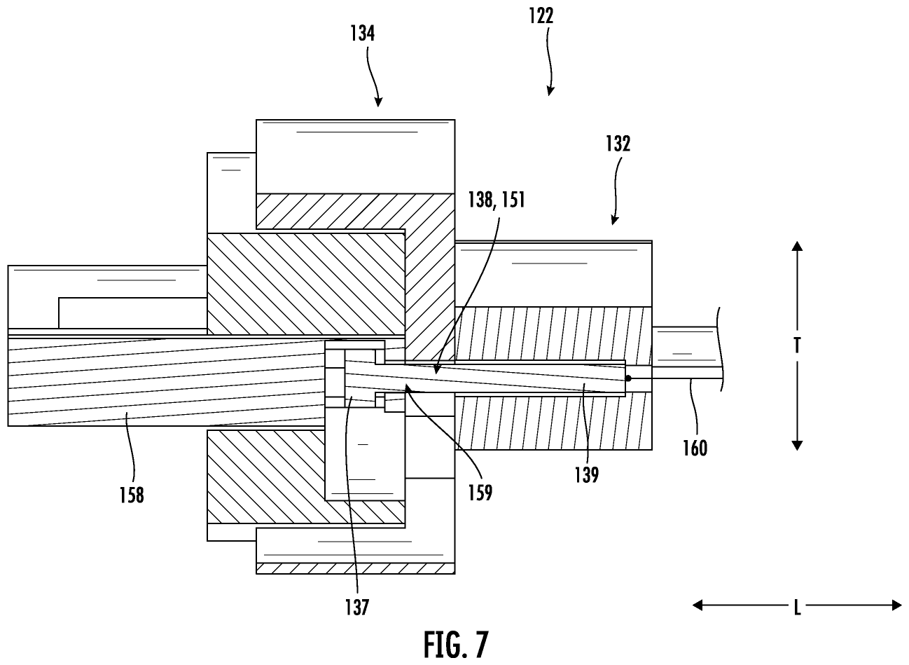

Referring back to FIG. 4, and now also to FIG. 7, which provides a close-up, schematic, cross-sectional view of the exemplary attachment assembly 122 of FIG. 4, taken along Line 7-7 in FIG. 4, FIG. 7 depicts the attachment assembly 122 in the attached configuration. The attachment assembly 122 further includes a translating component 158. The translating component 158 is connected to the control lever 126 on the handle of the diagnostic instrument 104 (see FIG. 3). The translating component 158 defines an opening 159 having a width greater than the thickness of the body 139 of the respective pin and less than the thickness of the distal end 125 of the respective pin. The widths and thicknesses are measured in the transverse direction T.

During operation of the diagnostic instrument 104, the translating component 158 is actuatable by the control lever 126, moving the translating component 158 along the longitudinal direction L relative to the first member 132 and the second member 134 of the attachment assembly 122. The pins are engaged with the penetrating component 120 to affect the operation of the distal end 125 of the penetrating component 120. Specifically, the diagnostic instrument 104 includes control wires 160, with each control wire 160 coupled to a respective pin, such that movement of the pins articulates the distal end 125 of the penetrating component 120 via the control wires 160.

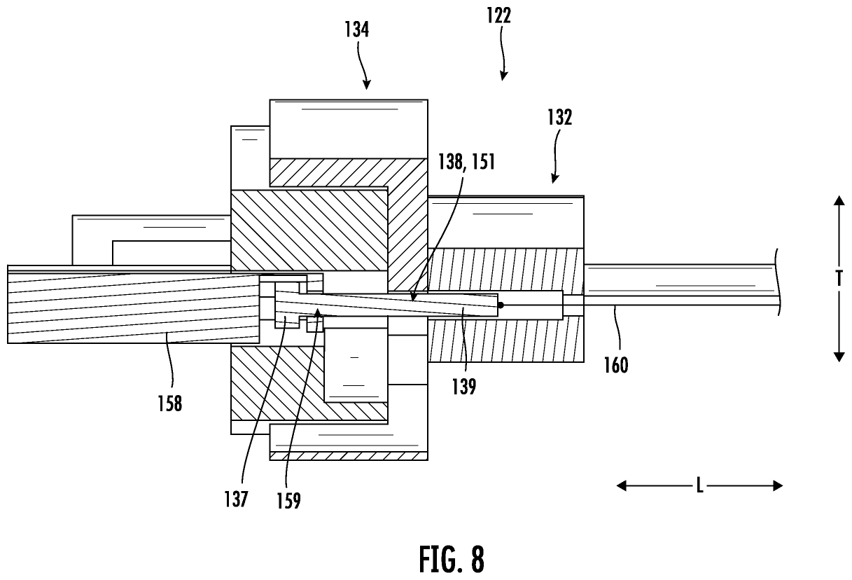

Referring now also to FIG. 8, the attachment assembly 122 of FIG. 7 is depicted with the translating component 158 moved from a first position (see FIG. 7) to a second position (see FIG. 8) along the longitudinal direction L. Movement of the translating component 158 along the longitudinal direction L from the first position to the second position correspondingly moves the control wire 160 coupled to the pin to affect operation of the distal end 125 of the penetrating component 120.

Referring briefly back to FIG. 4, the translating component 158 is a first translating component engaged with a first pin coupled to a first control wire 160, and the attachment assembly 122 further includes a second translating component 158 engaged with a second pin of the plurality of pins coupled to a second control wire 160 of the control wires 160. Operation of the first translating component 158 and the second translating component 158 may be affected by the control lever 126 of the diagnostic instrument 104 (see FIG. 3).

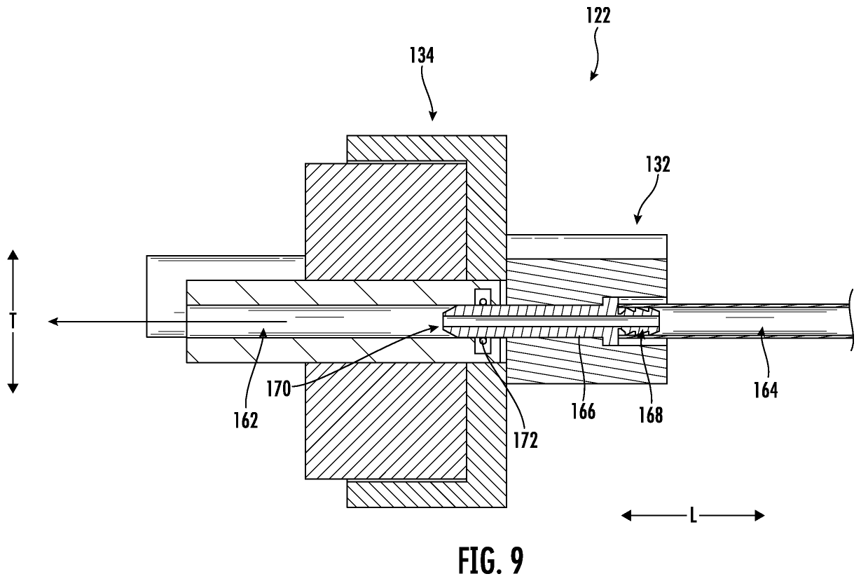

Referring now to FIG. 9, a schematic, cross-sectional view of the attachment assembly 122 of FIG. 4 is provided, taken along Line 9-9 in FIG. 4. The attachment assembly 122 is in the attached configuration in FIG. 9.

The proximal end 118 of the diagnostic instrument 104 defines a proximal end working channel 162 and the penetrating component 120 of the diagnostic instrument 104 defines a penetrating component working channel 164. The attachment assembly 122 includes a mating tube 166, with the proximal end working channel 162 being in communication with the penetrating component working channel 164 through the mating tube 166.

During operation of the diagnostic instrument 104, fluids (e.g., air, gas, oxygen), wires, cables, etc. may be provided through the proximal end working channel 162, mating tube 166, and penetrating component working channel 164, allowing for the flow of fluid (e.g., air, gas, oxygen), data, electrical power, etc. to the distal end 125 of the penetrating component 120 of the diagnostic instrument 104.

The mating tube 166 extends between a first end 168 and a second end 170. The first end 168 is fixedly coupled to the penetrating component working channel 164 through a friction fit connection. The second end 170 is slidably connected to the proximal end working channel 162. The attachment assembly 122 further includes a seal 172 located between the mating tube 166 and the proximal end working channel 162 to maintain a fluidic seal.

With this configuration, the mating tube 166 may be removed from the proximal end working channel 162 when the attachment assembly 122 is moved from the attached configuration to a detached configuration, and may be received within the proximal end working channel 162 when the attachment assembly 122 is moved from the detached configuration to the attached configuration.

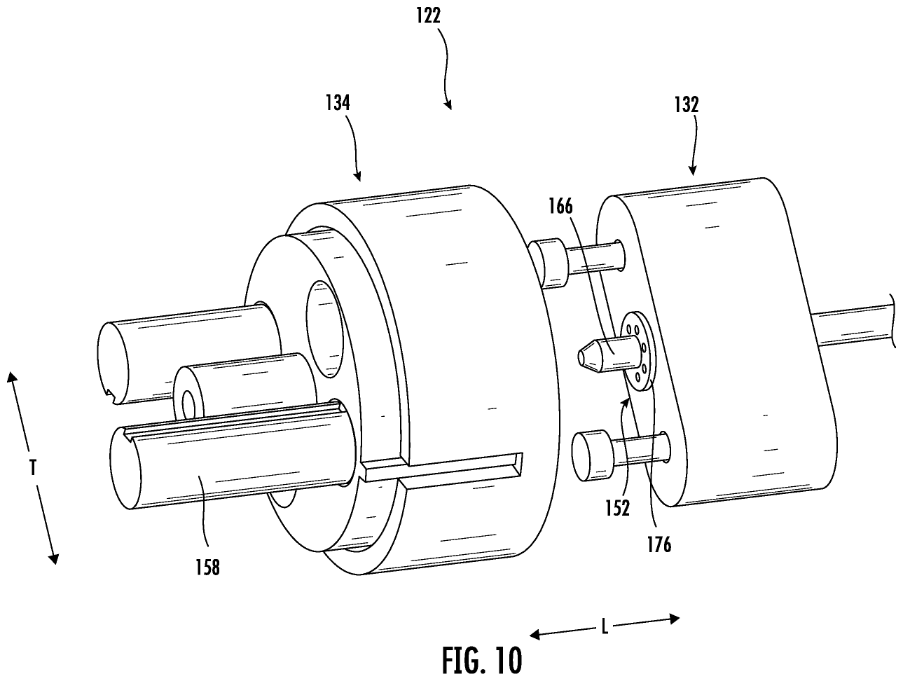

Referring back to FIG. 4 and now also to FIG. 10, providing another perspective view of the exemplary attachment assembly 122, it will be appreciated that the exemplary attachment assembly 122 further includes an electrical connection 152 allowing for communication between the first member 132 and the second member 134 of the attachment assembly 122. Specifically, the second member 134 includes electrical pins 174 extending along the longitudinal direction L towards the first member 132, and the first member 132 includes a pin receptacle 176 configured to receive the electrical pins 174 from the second member 134.

The electrical pins 174 may be spring-loaded, retracting along the longitudinal direction L once received within the pin receptacle 176. The pin receptacle 176 may include a printed circuit board or other suitable structure to receive and transmit power and/or data from to and/or from the electrical pins 174. The electrical connection 152 may therefore transmit power, data (e.g., video data), etc.

When the attachment assembly 122 is in the attached configuration, it may allow for fluidic communication between the penetrating component working channel 164 and the proximal end working channel 162, articulation of the distal end 125 of the penetrating component 120 through control wires 160, and other functionalities. The attachment assembly 122 may be moved to a detached configuration for certain operations without retracting the penetrating component 120.

Notably, in some embodiments reattachment may not be possible. Further, in some embodiments the reconnection of the proximal end to the penetrating component may not be possible. Further, in some embodiments reattachment is possible without retaining any functionality of the diagnostic instrument. Further, in some embodiments reattachment would be possible while retaining some functionality of the diagnostic instrument. Further, in some embodiments reattachment would be possible while retaining full functionality of the diagnostic instrument.

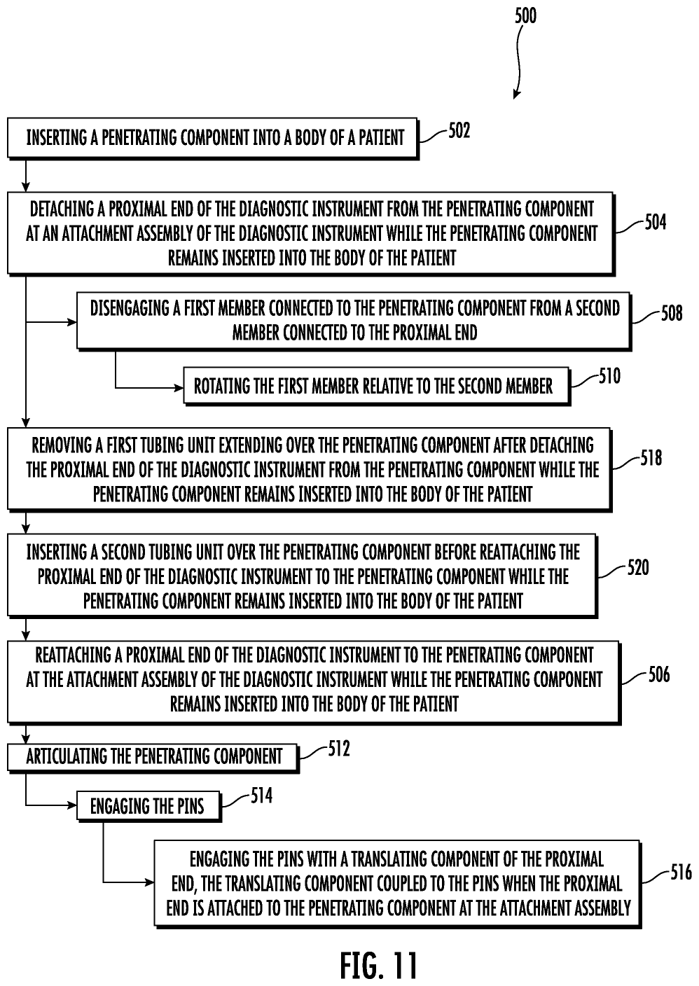

Referring now to FIG. 11, a flow diagram of a method 500 of operating a diagnostic instrument in accordance with an exemplary aspect of the present disclosure is provided.

In the exemplary aspect depicted, the method 500 includes at (502) inserting a penetrating component into a body of a patient; at (504) detaching a proximal end of the diagnostic instrument from the penetrating component at an attachment assembly of the diagnostic instrument while the penetrating component remains inserted into the body of the patient; and at (506) reattaching a proximal end of the diagnostic instrument to the penetrating component at the attachment assembly of the diagnostic instrument while the penetrating component remains inserted into the body of the patient.

An exemplary aspect depicted, detaching the proximal end of the diagnostic instrument from the penetrating component at the attachment assembly at (504) includes at (508) disengaging a first member connected to the penetrating component from a second member connected to the proximal end, and more specifically, at (510) rotating the first member relative to the second member (which includes rotating the first member while the second member stays stationary, rotating the second member while the first member stays stationary, or rotating both the first member and the second member). In such a manner, will be appreciated that the first member is rotatably engaged with the second member in the exemplary aspect depicted.

Notably, in at least certain exemplary aspects, the first member includes first attachment features, and the second member includes second attachment features. The first attachment features are, in at least certain exemplary aspects, pins with an enlarged distal end and the second attachment features are, in at least certain exemplary aspects, slots defined by the second member configured to receive the pins. Notably, with such exemplary aspect, the method may further include at (512) articulating the penetrating component, wherein articulating the penetrating component at (512) includes at (514) engaging the pins. In particular, engaging the pins at (514) includes at (516) engaging the pins with a translating component of the proximal end, the translating component coupled to the pins when the proximal end is attached to the penetrating component at the attachment assembly.

Further, as will be appreciated, the method 500 of FIG. 11 may allow for switching out a tubing unit without removal of the penetrating component from the body of the patient. In particular, for the exemplary aspect of the method 500 depicted in FIG. 11, the method 500 further includes at (518) removing a first tubing unit extending over the penetrating component after detaching the proximal end of the diagnostic instrument from the penetrating component while the penetrating component remains inserted into the body of the patient; and at (520) inserting a second tubing unit over the penetrating component before reattaching the proximal end of the diagnostic instrument to the penetrating component while the penetrating component remains inserted into the body of the patient. For the exemplary aspect depicted, the first tubing unit is different than the second tubing unit.

It will be appreciated that for the exemplary aspect of the method 500 depicted in FIG. 11 the first tubing unit may be an endotracheal tube in airflow communication with a ventilator.

As set forth above, it will be understood that the diagnostic instrument of the present invention may be any body-insertable diagnostic instrument and is not limited to an endoscope.

This written description uses examples to disclose the present disclosure, including an exemplary mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

We claim:

1. A diagnostic instrument comprising:
a proximal end;
a penetrating component; and
an attachment assembly positioned between the proximal end and the penetrating component to allow for separation of the proximal end from the penetrating component without fully retracting the penetrating component from a body of a patient during use of the diagnostic instrument.

2. The diagnostic instrument of claim 1, wherein the attachment assembly comprises a first member connected to the penetrating component and a second member connected to the proximal end, said first member and said second member releasably attachable to one another.

3. The diagnostic instrument of claim 2, wherein the first member includes first attachment features, and the second member comprises second attachment features, wherein the first and second attachment features interlock when rotated relative to each other.

4. The diagnostic instrument of claim 3, wherein the first attachment features comprise pins with an enlarged distal end and the second attachment features comprise slots defined by the second member configured to receive the pins.

5. The diagnostic instrument of claim 4, wherein the proximal end comprises a control lever and a translating component connected to the control lever, wherein the pins, when engaged, are actuatable by the control lever.

6. The diagnostic instrument of claim 5, wherein the pins are engaged with the penetrating component to articulate the penetrating component.

7. The diagnostic instrument of claim 1, wherein the proximal end defines a working channel, wherein the penetrating component defines a working channel, wherein the attachment assembly comprises a mating tube, and wherein the working channel of the proximal end is in communication with the working channel of the penetrating component through the mating tube.

8. The diagnostic instrument of claim 1, wherein the attachment assembly further comprises an electrical connection electrically connecting the proximal end and the penetrating component.

9. The diagnostic instrument of claim 1, wherein the diagnostic instrument is an endoscope.

10. A method of operating a diagnostic instrument, the method comprising:
inserting a penetrating component into a body of a patient;
detaching a proximal end of the diagnostic instrument from the penetrating component at an attachment assembly of the diagnostic instrument while the penetrating component remains inserted into the body of the patient; and
reattaching a proximal end of the diagnostic instrument to the penetrating component at the attachment assembly of the diagnostic instrument while the penetrating component remains inserted into the body of the patient.

11. The method of claim 10, wherein detaching the proximal end of the diagnostic instrument from the penetrating component at the attachment assembly comprises disengaging a first member connected to the penetrating component from a second member connected to the proximal end.

12. The method of claim 11, wherein disengaging the first member connected to the penetrating component from the second member connected to the proximal end comprises rotating the first member relative to the second member.

13. The method of claim 11, wherein the first member includes first attachment features, wherein the second member comprises second attachment features, wherein the first attachment features comprise pins with an enlarged distal end and the second attachment features defines slots configured to receive the pins.

14. The method of claim 13, further comprising:
articulating the penetrating component, wherein articulating the penetrating component comprises engaging the pins.

15. The method of claim 14, wherein engaging the pins comprises engaging the pins with a translating component of the proximal end, the translating component coupled to the pins when the proximal end is attached to the penetrating component at the attachment assembly.

16. The method of claim 10, further comprising:
removing a first tubing unit extending over the penetrating component after detaching the proximal end of the diagnostic instrument from the penetrating component while the penetrating component remains inserted into the body of the patient; and
inserting a second tubing unit over the penetrating component before reattaching the proximal end of the diagnostic instrument to the penetrating component while the penetrating component remains inserted into the body of the patient.

17. The method of claim 16, wherein the first tubing unit is different than the second tubing unit.

18. The method of claim 16, wherein the first tubing unit is an endotracheal tube in airflow communication with a ventilator.

19. A medical device assembly comprising:
a secondary device having a tube; and
a diagnostic instrument comprising:
a proximal end;
a penetrating component, the penetrating component extending at least partially through the tube of the secondary device; and
an attachment assembly positioned between the proximal end and the penetrating component to allow for separation of the proximal end from the penetrating component without fully retracting the penetrating component from a body of a patient during use of the diagnostic instrument.

20. The medical device assembly of claim 19, wherein the tube of the secondary device is an endotracheal tube, and wherein the diagnostic instrument is an endoscope.
